(19) United States
(12) Patent Application Publication
(10) Pub. No.: US 2012/0136367 A1
(43) Pub. Date: May 31, 2012

(54) MULTI-SEGMENT PROTECTIVE SHEATH FOR EXPANDABLE MEDICAL DEVICES

(75) Inventors: Stephen Pacetti, San Jose, CA (US); Binh T. Nguyen, Newark, CA (US)

(73) Assignee: Abbott Cardiovascular Systems, Inc., Santa Clara, CA (US)

(21) Appl. No.: 12/955,132

(22) Filed: Nov. 29, 2010

(51) Int. Cl. A61B 17/00

(52) U.S. Cl. 606/108

(57) ABSTRACT

A protective sheath for covering an elongated medical device includes two or more sheath segments. Each sheath segment has a lumen extending the length of the segment with an inner diameter sufficient to receive the elongated medical device within the lumen. Each sheath segment also has a first end and a second end, with the first end of each sheath segment being configured to engage the second end of an adjacent sheath segment in an interlocking engagement. The first end of each sheath segment has a first coupling portion and the second end of each sheath segment has a second coupling portion, which are complementary of one another and configured to couple adjacent sheath segments to one another when the first end of one sheath segment is engaged with the second end of an adjacent sheath segment.

### BACKGROUND OF THE INVENTION

I. The Field of the Invention

The present invention generally relates to the field of medical devices. More specifically, the present invention relates to protective sheaths for use with expandable medical devices.

II. Related Technology

Percutaneous coronary intervention (PCI) is a procedure for treating vascular disease. A catheter assembly having a balloon portion is introduced percutaneously into the cardiovascular system of a patient via the radial, brachial or femoral artery. The catheter assembly is advanced through the vasculature until the balloon portion is positioned across an occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure, placement of a stent, or a surgical by-pass operation.

One approach to prevent the thrombosis and restenosis associated with the use of the balloons is to coat a drug or other beneficial agent onto the balloon. When the balloon is deployed, the drug or beneficial agent is delivered to the vessel wall to prevent or mitigate thrombosis and/or restenosis. Often the drug is coated onto the balloon during a manufacturing process, folded into a small profile, and then covered with a protective sheath. The purpose of this sheath is to keep the folded balloon at a low profile as well as to protect the balloon and drug coating until the device is ready for use. However, friction between the protective sheath and the balloon as the protective sheath is placed in position over the balloon, or when it is removed, can damage the drug coating and/or the balloon.

### BRIEF SUMMARY OF THE INVENTION

In one aspect of the disclosure, a protective sheath for an elongated medical device includes two or more sheath segments, each sheath segment having a lumen extending the length of the segment with an inner diameter sufficient to receive the elongated medical device within the lumen.

In another aspect that may be combined with any of the aspects herein, each sheath has a first end and a second end, the first end of each sheath segment being configured to engage the second end of an adjacent sheath segment in interlocking engagement.

In another aspect that may be combined with any of the aspects herein, the first end of each sheath segment has a first coupling portion, and the second end of each sheath segment has a second coupling portion.

In another aspect that may be combined with any of the aspects herein, the first and second coupling portions are complementary of one another and configured to couple adjacent sheath segments to one another when the first end of one sheath segment is engaged with the second end of an adjacent sheath segment.

In another aspect that may be combined with any of the aspects herein, the first coupling portion includes external coupling features and the second coupling portion includes interior coupling features.

In another aspect that may be combined with any of the aspects herein, the external coupling features include at least one of ridges, teeth, or threads.

In another aspect that may be combined with any of the aspects herein, the interior coupling features includes ridges, teeth, or threads complimentary to the ridges, teeth, or threads associated with the external coupling features.

In another aspect that may be combined with any of the aspects herein, the external coupling features include at least one ramped tab.

In another aspect that may be combined with any of the aspects herein, the interior coupling features includes at least one annular groove.

In another aspect that may be combined with any of the aspects herein, the sheath segments comprise poly(ethylene), poly(propylene), poly(olefins), poly(tetrafluoroethylene), poly(tetrafluoroethylene-co-hexafluoropropylene), poly(tetrafluoroethylene-co-ethylene), poly(vinylidene fluoride), poly(vinylidene fluoride-co-hexafluoropropylene), fluoropolymers, nylon-12, nylon-6, nylon-6,6, nylons, polyesters, or blends or coextrusions thereof

In another aspect that may be combined with any of the aspects herein, the elongated medical device comprises a drug coated balloon.

In another aspect that may be combined with any of the aspects herein, an expandable medical device includes an expandable member and a protective sheath covering said expandable member.

In another aspect that may be combined with any of the aspects herein, the protective sheath includes a first sheath segment and a second sheath segment, the first sheath segment and the second sheath segment being configured to be positioned separately over the expandable member and to be removed together.

In another aspect that may be combined with any of the aspects herein, the first sheath segment and the second sheath segment are configured to be sealingly coupled together to cover the expandable member

In another aspect that may be combined with any of the aspects herein, the first sheath segment and the second sheath segment are configured to be sealingly coupled by pushing the second sheath segment against the first sheath segment.

In another aspect that may be combined with any of the aspects herein, the expandable medical device includes at least one ramped tab associated with the first sheath segment and an annular groove associated with the second sheath segment, the ramped tab being configured to engage the annular groove

In another aspect that may be combined with any of the aspects herein, the first sheath segment and the second sheath segment are configured to be rotated into sealing engagement.

In another aspect that may be combined with any of the aspects herein, the expandable medical device includes a beneficial agent loaded onto a surface of the expandable member.

In another aspect that may be combined with any of the aspects herein, the first segment has a first end having a first diameter and the second segment includes a second end having a second diameter, the first diameter being smaller than the second diameter so as to allow the first end to be received in the second end.

In another aspect that may be combined with any of the aspects herein, a protective sheath includes a plurality of sheath segments configured to cover a portion of an expandable member, each sheath segment having a length that is less than a length of the expandable member.

In another aspect that may be combined with any of the aspects herein, each sheath segment includes a first end, a second end, and a lumen defining a conduit between said first end and second end.

In another aspect that may be combined with any of the aspects herein, a first region of the lumen associated with the first end has a first diameter and a second region of the lumen associated with the second end has a second diameter, the second diameter being greater than the first diameter.

In another aspect that may be combined with any of the aspects herein, each sheath segment further includes first coupling features and second coupling features, the second coupling features being in communication with the lumen and the first coupling features being positioned on an outer portion of the sheath segment.

In another aspect that may be combined with any of the aspects herein, the first coupling features include at least one of ridges, teeth, and threads.

In another aspect that may be combined with any of the aspects herein, the second coupling features include at least one of ridges, teeth, and threads.

In another aspect that may be combined with any of the aspects herein, the first coupling features include ramped tabs and the second coupling features include an annular groove.

In another aspect that may be combined with any of the aspects herein, a method includes providing an expandable medical device having an expandable member and coating a beneficial agent onto the expandable member.

In another aspect that may be combined with any of the aspects herein, the method includes positioning a first sheath segment over the expandable member to cover a first portion of the expandable member.

In another aspect that may be combined with any of the aspects herein, the method includes separately positioning a second sheath segment over the expandable member to cover a second portion of the expandable member and to couple the second sheath segment to the first sheath segment such that the first sheath segment and the second sheath segment form a protective sheath over the expandable member and the beneficial agent.

In another aspect that may be combined with any of the aspects herein, the method includes separately positioning a third sheath segment over the expandable member and coupling the third sheath segment to the second sheath segment.

In another aspect that may be combined with any of the aspects herein, the method includes sterilizing the expandable medical device after the first sheath segment and the second sheath segment are positioned over the expandable member.

In another aspect that may be combined with any of the aspects herein, positioning the first sheath segment over the expandable member includes passing a proximal end of the first sheath segment over a distal end and a proximal end of the expandable member such that a distal end of the first sheath segment is positioned between the distal end and the proximal end of the expandable member.

In another aspect that may be combined with any of the aspects herein, the protective sheath is removable from the expandable member as a single piece after the first sheath segment is coupled to the second sheath segment.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A illustrates a side view of an expandable medical device with a multi-segment protective sheath positioned thereon according to one example;

FIG. 1B illustrates a side view of a expandable medical device with the multi-segment protective sheath removed according to one example;

FIG. 2A illustrates a perspective view of an individual sheath segment of a multi-segment protective sheath according to one example;

FIG. 2B illustrates the individual sheath segment in position on a expandable medical device according to one example;

FIG. 2C illustrates a plurality of sheath segments of a multi-segment protective sheath in position on an expandable medical device according to one example;

FIG. 2D illustrates a multi-segment protective sheath in position on an expandable medical device;

FIG. 2E illustrates a cross-sectional view of the multi-segment protective sheath in position on the balloon catheter taken along section 2-2 of FIG. 2D;

FIGS. 3A-3B illustrate a method of crossing an obstruction in a body lumen using an expandable medical device according to one example.

FIG. 4A illustrates a perspective view of an individual sheath segment of a multi-segment protective sheath according to one example; and

FIG. 4B illustrates a cross-sectional view of a multi-segment protective sheath.

Together with the following description, the figures demonstrate and explain the principles of protective sheath for use with expandable medical devices, including drug coated balloons. In the figures, the thickness and configuration of components can be exaggerated for clarity. The reference numerals in different figures represent similar, though not necessarily identical, components.

### DETAILED DESCRIPTION

Multi-segment protective sheaths are provided herein that each include a plurality of individual sheath segments that are applied sequentially to cover an expandable medical device. The individual sheath segments couple together such that when a protective sheath is removed from the expandable medical device, the sheath segments are removed together as one overall sheath. For ease of reference, the expandable medical device discussed below will be shown as a balloon catheter, though it will be appreciated that the protective sheaths may be utilized with any number of expandable medical devices. Further, in the examples described below, the protective sheaths will be described in conjunction with balloon catheters that have been coated with a beneficial agent. Various beneficial agents and methods of applying beneficial agents to balloon catheters will be described herein.

Balloon catheters intended for dilatation of vessels are equipped with non-compliant or semi-compliant balloons. These balloons enable expansion of the lesion with pressure without excessive axial growth of the balloon. Such balloons are folded in a deflated state. A key performance parameter of a balloon is its diameter, or profile, in the deflated state. It is desired that the profile be small to facilitate delivery of the balloon through tortuous anatomy and tight lesions. To maintain this small profile, after the balloons are folded to a small diameter, a snug sheath is placed over the balloon to maintain this small profile on storage. Without the snug, protective sheath, the balloon folds will relax with time and the diameter will increase. Hence, the protective sheath on a balloon, including a balloon coated with a beneficial agent, has two functions. One is to protect the drug coating during manufacturing, sterilization, shipping, and storage, and the second function is to preserve the small diameter of the folded balloon.

In the case of a balloon catheter that is coated with beneficial agents, a protective sheath that includes multiple sheath segments may allow the protective sheath to be applied in such a manner as to minimize damage of the balloon and/or the coating of the beneficial agent both as the protective sheath is positioned on the balloon as well as after the protective sheath is in place over the balloon. For example, in some instances, applying a single, long sheath to a balloon results in so much friction that the proximal section of the balloon buckles before the sheath is completely on, damaging the balloon and/or coating. This buckling may be reduced by grasping the balloon where buckling occurs to support the balloon and enable the sheath to be pushed on. However, grasping a balloon with a drug coating can damage the coating, resulting in reduced drug dosage intended for the balloon as well as potential for contamination.

Positioning each of multiple sheath segments of a protective sheath individually can result in less friction between the individual sheath segments and the balloon than would be associated with applying a single sheath that extends the entire length of the balloon. As multiple sheath segments are positioned on the balloon, each sheath segment couples to a previously positioned sheath segment until the entire protective sheath is in position on the balloon. With the desired number of sheath segments thus in place, the protective sheath covers the entire balloon, thus protecting the balloon from damage and/or contamination during shipping or other processes.

Once a practitioner desires to deploy the balloon catheter, the entire protective sheath may be removed from the balloon as a single unit, thereby facilitating easy removal of the sheath. This easy, one step removal of the sheath is advantageous as it minimizes damage to the coating. If the individual sections were not connected then they would have to be removed one at a time. This greater number of manipulations increases the likelihood of the operator touching or damaging the coating.

The sheath itself may be composed of a variety of polymeric materials. Typical sheath materials include poly(ethylene), poly(propylene), poly(olefins), poly(tetrafluoroethylene), poly(tetrafluoroethylene-co-hexafluoropropylene), poly(tetrafluoroethylene-co-ethylene), poly(vinylidene fluoride), poly(vinylidene fluoride-co-hexafluoropropylene), fluoropolymers, nylon-12, nylon-6, nylon-6,6, nylons, polyesters and blends or coextrusions of the above.

The overall configuration and function of an expandable medical device will first be discussed to provide an exemplary context. Thereafter, the configuration of an exemplary multi-segment protective sheath will be discussed in more detail, including a discussion of placement of the protective sheath on an exemplary expandable medical device. Beneficial agents that may be used on the expandable medical device in conjunction with the multi-segment protective sheath will then be discussed, followed by a discussion of the use of the expandable medical device and protective sheath in a medical procedure.

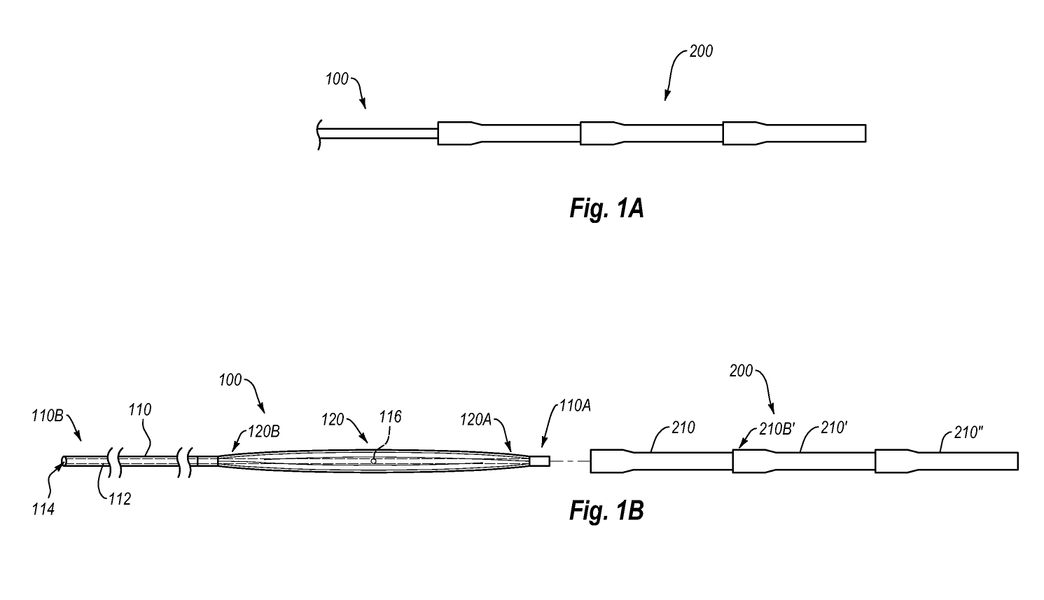

FIG. 1A illustrates a side view of an expandable medical device 100, such as a balloon catheter, with a multi-segment protective sheath 200 (also referred to simply as a protective sheath) positioned thereon according to one example. FIG. 1B illustrates a side view of the expandable medical device 100 in which the protective sheath 200 has been removed. As illustrated in FIG. 1B, the balloon catheter 100 generally includes a shaft 110 having a distal end 110A and a proximal end 110B. An expandable member 120, such as an expandable balloon, may be positioned adjacent the distal end 110A of the shaft 110. The protective sheath 200 may be positioned on the expandable member 120 to protect the expandable member 120 and any coatings or substances applied thereto from damage and/or contamination.

In at least one example, the shaft 110 may include a lumen 112 defined therein that extends from the proximal end 110B toward the distal end 110A. The lumen 112 may provide fluid communication between an opening 114 defined in or adjacent the proximal end 110B of the shaft 100 and one or more opening 116 in the shaft 110 in the region of the expandable member 120. In such a configuration, fluid introduced to the opening 114 in the proximal end 110B is directed through the lumen 112 to the opening 116 and to the expandable member 120.

The expandable member 120 may include a distal end 120A and proximal end 120B that are each sealingly coupled to the shaft 110. As a result, the fluid directed through the opening 116 to the expandable member 120 may act to cause the expandable member 120 to expand. As will be described in a more appropriate location hereinafter, the expansion of the expandable member 120 can allow the expandable member 120 to open an obstruction or occlusion at a deployment site in a body lumen. Further, the expansion of the expandable member 120 may allow the expandable member 120 to deliver beneficial agents to the deployment site. In particular, beneficial agents may be applied to the outer surface of the expandable member 120. The application of exemplary beneficial agents will be described in more detail at an appropriate point hereinafter. In at least one example, the beneficial agents may be applied to the expandable member 120 as part of a process that is performed before the balloon catheter 100 is shipped to a health care provider for use in a medical procedure. The beneficial agent and/or the expandable member 120 may be damaged and/or contaminated during handling after the beneficial agent has been applied, which would reduce the efficacy of the sheath, catheter, and/or the beneficial agent.

Accordingly, the protective sheath 200 described in hereinafter may cover the expandable member 120 after the beneficial agent is applied to the expandable member 120 to protect the expandable member 120 and/or the beneficial agent from contamination and/or damage after the beneficial agent has been applied. In at least on example, the protective sheath 200 is configured to be positioned over the beneficial agent and the expandable member 120 with a relatively low amount of friction. Maintaining a relatively low amount of friction between the expandable member 120 and the protective sheath 200 may reduce damage to the expandable member 120 and/or the beneficial agent as the protective sheath 200 is positioned over the expandable member 120.

In one embodiment, the protective sheath 200 may include a plurality of individual sheath segments 210, 210′, 210″ that are sequentially positioned over the expandable member 120 to protect the beneficial agent and/or the expandable member 120 from contamination and/or damage as well as to help maintain the beneficial agent in place on the expandable member 120. After an individual sheath segment 210 has been positioned over the expandable member 120, when subsequent individual sheath segments 210′, 210″ are positioned over the expandable member 120, the subsequent individual sheath segments 210′, 210″ couple to the previously positioned individual sheath segment(s) 210, (210′) to thereby form the continuous protective sheath 200.

The frictional force between the protective sheath 200 and the expandable member 120 as the protective sheath 200 is installed depends, at least in part, on the total surface area of the protective sheath 200 that is in contact with the expandable member 120 as the protective sheath 200 is moved into position. Since each of the individual sheath segments 210, 210′, 210″ represents only a portion of the length of the entire protective sheath 200, the frictional force between each of the individual sheath segments 210, 210′, 210″ and the expandable member 120 is a fraction of what would be associated with positioning all of the individual sheath segments 210, 210′, 210″ at the same time. Exemplary multi-segment protective sheaths and methods of placing the protective sheaths over expandable members will now be discussed in more detail.

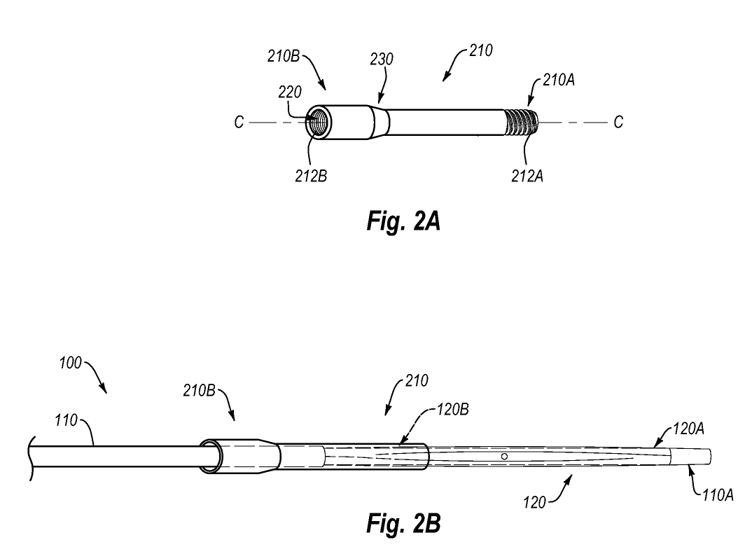

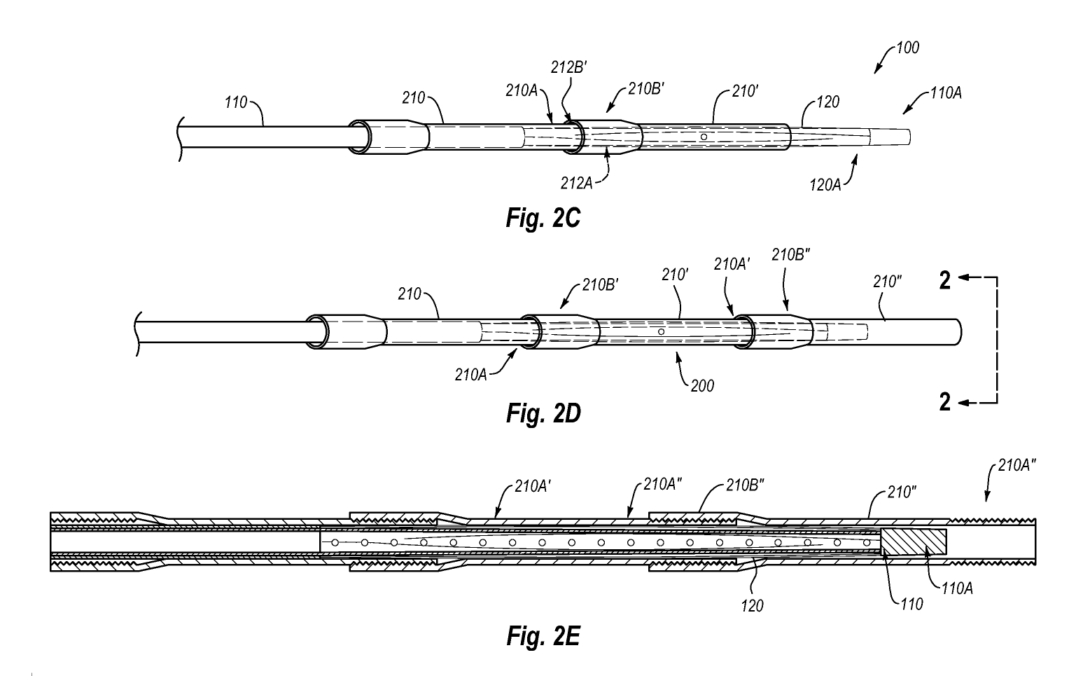

FIG. 2A illustrates a perspective view of an individual sheath segment 210 of the multi-segment protective sheath 200 (FIG. 1) according to one example. The sheath segment 210 may be substantially similar as the other individual sheath segments 210′, 210″ (FIG. 1B) used to form the protective sheath 200 or the single sheath segment 210 may be different than other sheath segments 210′, 210″. For ease of reference, the single sheath segment 210 will be described as being substantially similar to individual sheath segments 210′, 210″ (FIG. 1A).

In the illustrated example, the individual sheath segment 210 generally includes a distal end 210A and a proximal end 210B. These designations are provided for ease of reference only and it will be appreciated that the orientation may be switched as desired. A lumen 220 is defined in the individual sheath segment 210 that passes between the distal end 210A and the proximal end 210B thereby causing the lumen 220 to define a conduit through the individual sheath segment 210. As a result, the individual sheath segment 210 may be described as having an inner surface in communication with the lumen 220. The individual sheath segment 210 may further be described as having an outer surface, the outer surface being opposite the inner surface.

As show in FIG. 2A, each sheath segment 210 also has a distal coupling feature 212A and a proximal coupling feature 212B. Distal coupling feature 212A and proximal coupling feature 212B are complimentary of one another, so that the distal coupling feature 212A of one sheath segment, such as sheath segment 210 (FIG. 1B), may be selectively and securely coupled to the proximal coupling feature 212B of an adjacent sheath segment, such as sheath segment 210′ (FIG. 1B) in an interlocking arrangement.

In at least one example, the distal coupling features 212A associated with the distal end 210A can be formed on the outer surface of the individual sheath segment near the distal end 210A. These distal coupling features 212A can have any desired configuration, such as exterior ridges or teeth as shown in FIG. 2A, or threads. As introduced, the distal coupling features 212A are configured to couple to a proximal end 210B′ of the adjacent individual sheath segment 210′ (both shown in FIG. 1B), which may be configured similarly as the proximal end 210B of the individual sheath segment 210 shown. Thus, the coupling features 212B may be interior ridges, teeth or threads.

As shown in FIG. 2A, a portion of the lumen 220 associated with the proximal coupling features 212B may have a larger width than a portion of the lumen 220 associated with the distal coupling features 212A. It will be appreciated that the lumen 220 can have any cross-sectional shape desired and that the cross sectional shape may vary as desired. For ease of reference, the individual sheath segment 200 will be described as having generally circular cross-sectional shapes along a central axis C-C of the individual sheath segment 200 and thus the widths may be described as diameters.

In at least one example, a transition region 230 may be provided between the distal end 210A and the proximal end 210B as desired to provide a transition between the diameter of the lumen 220 adjacent the proximal coupling features 212B and the diameter of the lumen 220 adjacent the distal coupling features 212A. In at least one example, the transition region 230 may cause the diameter of the lumen 220 to taper smoothly between the diameters described above or may taper in a step-wise or other fashion as desired. A smooth taper may allow the individual sheath segments 210 to readily receive an expandable member in the proximal end 210B, as will be discussed in more detail.

FIG. 2B illustrates the individual sheath segment 210 in position on the expandable medical device 100. In order to move the individual sheath segment 210 into the position shown, the proximal end 210B of the individual sheath segment 210 may be passed over the expandable member 120. In particular, the proximal end 210B of the individual sheath segment 210 may be pushed over the distal end 110A of the shaft 110 as well as the distal end 120A of the expandable member 120 to thereby position the proximal end 210B on the shaft 110 at a location that is proximal of the proximal end 120B of the expandable member 120. In at least one example, the individual sheath segment 210 may be sealingly coupled to the shaft 110 and/or the expandable member 120, thereby providing a seal from contamination through the proximal end 210B of the individual sheath segment 210.

As shown in FIG. 2C, an additional individual sheath segment 210′ may be positioned over the expandable member 120. In particular, the additional individual sheath segment 210′ may be positioned on the expandable medical device 100 by passing a proximal end 210B′ of the individual sheath segment 210′ over the distal end 110A of the shaft 110 as well as the distal end 120A of the expandable member 120. Passing the proximal end 210B′ further in the proximal direction allows the proximal end 210B′ to sealingly couple to the distal end 210A. Consequently, proximal coupling features 212B′ associated with the adjacent individual sheath segment 210′, which are interior features, may be configured to couple to the distal coupling features 212A of the individual sheath segment 210, which are exterior features. In at least one example, the proximal coupling features 212B′ pass onto the distal coupling features 212A as the adjacent individual sheath segment 210′ is moved into proximity with the individual sheath segment 210.

In at least one example, sealing engagement between the proximal end 210B′ and the distal end 210A may be established by relative rotation between the proximal end 210B′ and the distal end 210A. In other examples, axial translation of the proximal end 210B′ relative the shaft 110 may be sufficient to cause the proximal end 210B′ to sealingly couple to the distal end 210A through an interference fit.

Referring briefly to FIG. 2D, a proximal end 210B″ of the individual sheath segment 210″ may be coupled to the distal end 210A′ of the individual sheath segment 210′ in a manner similar to that described for coupling the proximal end 210B′ of the individual sheath segment 210′ to the distal end 210A of the individual sheath segment 210 as shown in FIG. 2C. In such an example, once the proximal end 210B″ has been coupled to the distal end 210A′, the individual sheath segments 210, 210′, 210″ form the protective sheath 200.

Referring now to FIG. 2E, in at least one example, when the proximal end 210B″ of the individual sheath segment 210″ is coupled to the distal end 210A′, a distal end 210A″ of the individual sheath segment 210″ may extend beyond a distal end 110A of the shaft 110. Such a configuration allows a practitioner to grasp the distal end 210A″ without causing the distal end 210A″ to compress against the expandable member 120. Allowing the distal end 210A″ to compress without compressing the expandable member 120 may reduce damage to a coating of beneficial agent applied to the expandable member 120 during sheath removal. Examples of preparing expandable medical devices with beneficial agents and protective sheaths will now be discussed in more detail.

Referring again briefly to FIG. 1B, the underlying structure of the expandable member 120 can be virtually any structural design. Further, the balloon can be formed of any suitable material such as, but not limited to, polyester, PTFE (Teflon), nylon, Pebax® (Colombes Cedex, France), Dacron, poly(ethylene), or combinations thereof. “Teflon” and “Dacron” are understood to be trade names for polymers available from DuPont Co., Wilmington, Del. In some embodiments, the surface of the expandable member 120 can include one or more reservoirs or cavities formed therein or ports for solution delivery.

The shaft 110 and the expandable member 120 can be fabricated utilizing any number of methods known in the art. For example, the shaft 110 can be fabricated from a hollow or formed tube as described above. The expandable member 120 may be thin membranes of polymer that is solution or physically (by laser or ultrasonically) welded to the tube. The inner volume of the expandable member 120 is then in direct contact with the shaft 110 such that air or aqueous solutions can be injected into the space under pressure to expand the expandable member 120 into any predefined shape that is of use. The surface of the expandable member 120 can be rolled, pleated, or folded to reduce the outer diameter of the final expandable medical device 100.

The expandable member 120 can be in an expanded or unexpanded state during the loading of beneficial agent. Additionally, the expandable member 120 can be in a rolled, unrolled, pleated, or folded state during the loading of beneficial agent. The expandable member 120 can be loaded with one or more beneficial agent. “Beneficial agent” as used herein, refers to any compound, mixture of compounds, or composition of matter consisting of a compound, which produces a beneficial or useful result. The beneficial agent can be a polymer, a marker, such as a radiopaque dye or particles, or can be a drug, including pharmaceutical and beneficial agents, or an agent including inorganic or organic drugs without limitation.

The beneficial agents or drug can be in various forms such as uncharged molecules, components of molecular complexes, pharmacologically-acceptable salts such as hydrochloride, hydrobromide, sulfate, laurate, palmitate, phosphate, nitrate, borate, acetate, maleate, tartrate, oleate, and salicylate.

An agent or drug that is water insoluble can be used in a form that is a water-soluble derivative thereof to effectively serve as a solute, and on its release from the device, is converted by enzymes, hydrolyzed by body pH, or metabolic processes to a biologically active form. Additionally, the beneficial agents or drug formulations can have various known forms such as solutions, dispersions, pastes, particles, granules, emulsions, suspensions and powders. The drug or beneficial agent may or may not be mixed with polymer or a solvent as desired.

For purposes of illustration and not limitation, the drug or beneficial agent can include antithrombotics, anticoagulants, antiplatelet agents, thrombolytics, lipid-lowering agents, paclitaxel, protaxel, sirolimus, everolimus, zotarolimus, biolimus, myolimus, novolimus, deforolimus, antiproliferatives, anti-inflammatories, agents that inhibit hyperplasia, inhibitors of smooth muscle cell proliferation, antibiotics, growth factor inhibitors, cell adhesion promoters, or cell adhesion inhibitors. Other drugs or beneficial agents include but are not limited to antineoplastics, antimitotics, antifibrins, antioxidants, agents that promote endothelial cell recovery, antiallergic substances, radiopaque agents, viral vectors, antisense compounds, oligionucleotides, cell permeation enhancers, angiogenesis agents, and combinations thereof.

The beneficial agent can also include at least one biologically active (“bioactive”) agent. The at least one drug bioactive agent can include any substance capable of exerting a therapeutic, prophylactic or diagnostic effect for a patient. As used herein, the term drug and bioactive agent are used interchangeably.

Examples of suitable bioactive agents include, but are not limited to, synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. In certain embodiments, optionally in combination with one or more other embodiments described herein, the beneficial agent can include at least one biologically active agent selected from antiproliferative, antineoplastic, antimitotic, anti-inflammatory, antiplatelet, anticoagulant, antifibrin, antithrombin, antibiotic, antiallergic and antioxidant substances.

If desired, the beneficial agent may also include a binder to carry, load, or allow sustained release of an agent, such as but not limited to a suitable polymer or similar carrier. The term “polymer” is intended to include a product of a polymerization reaction inclusive of homopolymers, copolymers, terpolymers, etc., whether natural or synthetic, including random, alternating, block, graft, branched, cross-linked, blends, compositions of blends and variations thereof. The polymer may be in true solution, saturated, or suspended as particles or supersaturated in the beneficial agent. The polymer can be biocompatible, or biodegradable.

The beneficial agent can include a solvent. The solvent can be any single solvent or a combination of solvents. For purpose of illustration and not limitation, examples of suitable solvents include water, aliphatic hydrocarbons, aromatic hydrocarbons, methanol, ethanol, isopropanol, alcohols, acetone, ketones, dimethyl sulfoxide, tetrahydrofuran, dihydrofuran, dimethylacetamide, methyl acetate, ethyl acetate, esters, and combinations thereof.

A number of methods can be used to load the beneficial agent onto the surface of the expandable member 120 to provide for a controlled local area density of beneficial agent. For example, the expandable member 120 can be constructed to include pores or reservoirs which are impregnated or filled with beneficial agent or multiple beneficial agents. The pores can be sized or spaced apart to correspond to or limit the amount of beneficial agent contained therein in accordance with the desired local area density pattern along the length of the interventional device, wherein larger pores or more dense spacing would be provided in such portions intended to have a greater local area density. Alternatively, uniform pores sizes can be provided but the amount of beneficial agent loaded therein is limited accordingly. Additionally, if desired, a membrane of biocompatible material can then be applied over the pores or reservoirs for sustained or controlled release of the beneficial agent from the pores or reservoirs.

According to some of the embodiments, the beneficial agent can be loaded directly onto the expandable member 120 or alternatively, the beneficial agent can be loaded onto a base material layer that is applied to a surface of the expandable member 120. For example and not limitation, a base coating, such as a binder, primer, or suitable polymer, can be applied to a selected surface of the expandable member 120 such that a desired pattern is formed on the expandable member 120 surface. Beneficial agents may then be then applied directly to the pattern of the base material.

In yet another example, the beneficial agent can be applied directly to the surface of the expandable member 120. A binder or similar component can be used to help ensure sufficient adhesion. For example, this coating technique can include mixing the beneficial agent with a suitable binder or polymer to form a coating mixture, which is then coated onto the surface of the expandable member 120. The coating mixture can be prepared in higher or lower concentrations of beneficial agent as desired, and then applied to selected portions of the expandable member 120 appropriately.

In any of the embodiments disclosed herein, a porous or biodegradable membrane or layer made of biocompatible material can be coated over the beneficial agent for sustained release thereof, if desired.

Conventional coating techniques can be utilized to coat the beneficial agent onto the surface of the expandable member 120 such as spraying, brushing, wiping, direct fluid application, inkjet printing, roll coating, dipping or sputtering. With such techniques, it may be desirable or necessary to use known masking or extraction techniques to control the location and amount of beneficial agent loaded. Although not required, prior to coating the expandable member 120 with beneficial agent, optical machine vision inspection of the expandable member 120 may be utilized to ensure that no mechanical defects exist. Defective prostheses or balloons may be rejected before wasting beneficial agent, some of which may be very costly.

After the beneficial agent has been coated onto the expandable member 120 as described above, the protective sheath 200 may be applied by positioning the individual sheath segments 210, 210′, 210″ on the expandable medical device 100 as described above. Thereafter, with the protective sheath 200 in place, the expandable medical device 100 can be sterilized and packaged.

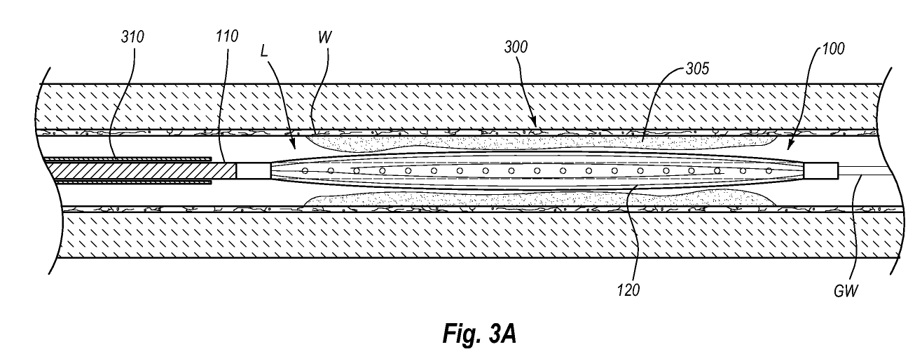

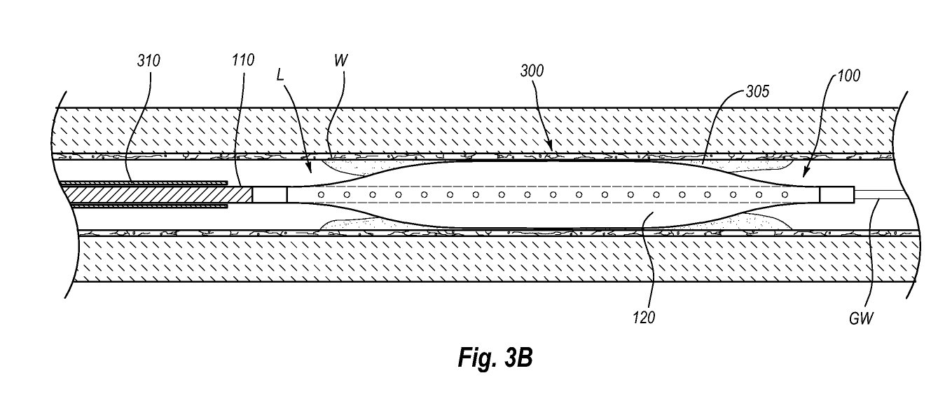

The expandable medical device 100 can then be provided to a medical practitioner for use in a medical procedure. One such medical procedure is shown in more detail in FIGS. 3A-3B. As shown in FIG. 3A, the procedure can begin by advancing a guidewire GW into proximity with a deployment site 300 within a body lumen L defined by a lumen wall W. In at least one example, the deployment site 300 may be the location of an obstruction 305 that constricts the lumen L.

Once the guidewire GW is in place, the protective sheath 200 (FIG. 2D) can be removed from the expandable medical device 100. The expandable medical device 100 can then be positioned within a guide catheter 310. The expandable medical device 100 and the guide catheter 300 can be advanced over the guidewire GW and into proximity with the deployment site 300. The expandable medical device 100 can then be advanced distally relative to the guide catheter 310 to position the expandable member 120 across the obstruction 305.

As shown in FIG. 3B, fluid may be provided to the expandable member 120 to cause the expandable member 120 to expand and exert a compressive force on the obstruction 305. The compressive force may be sufficient to open the obstruction 305 once the expandable medical device 100 has been removed. Further, as the expandable member 120 exerts the compressive force on the obstruction 305, the beneficial agent coated onto the expandable member 120 is delivered to the deployment site 300 through contact and the mechanisms described above.

The beneficial agent may help reduce restenosis associated with the process after the expandable member 120 is removed. In at least one example, the fluid described above may be withdrawn to allow the expandable member 120 to collapse. Thereafter, the expandable medical device 100 may be withdrawn proximally relative to the guide catheter 310 to draw the expandable member 120 into the guide catheter 305. The guide catheter 310, the expandable medical device 100, and the guidewire GW can then be removed to finish the procedure.

As discussed above with reference to FIG. 2A, the individual sheath segment 210 includes distal coupling features 210A and proximal coupling features 210B that cooperate with other coupling features to couple the individual sheath segment 210 to adjacent sheath segments, such as individual sheath segment 210′. The distal coupling features 210A and proximal engagement 210B described above included a plurality of ridges or threads that were configured to be threaded or pushed into engagement with other coupling features.

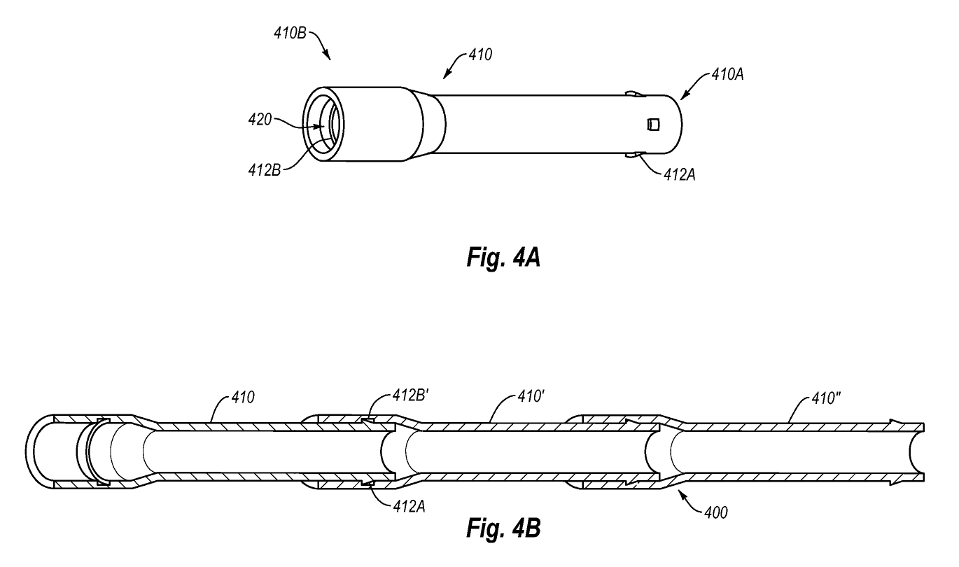

FIG. 4A illustrates a perspective view of an individual sheath segment 410 having an alternative configuration. As shown in FIG. 4A, the individual sheath segment 410 may generally include a distal end 410A and proximal end 410B with a lumen 420 defined therein in a similar manner as described above with individual sheath segment 210 (FIG. 2A). However, as shown in FIG. 4A, the individual sheath segment 410 may include ramped tabs 412A that extend outwardly from the distal end 410A. The individual sheath segment 410 may also include an annular groove 412B associated with the proximal end 410B.

As shown in FIG. 4B, the ramped tabs 412A may be configured to engaged an annular groove 412B′ in an adjacent individual sheath segment 410′ as shown in FIG. 4B. Similar engagement may couple segment 410″ to segment 410. In such an example, the ramped tabs 412A and other similar features may facilitate coupling of the individual sheath segments 410, 410′, 410″ together to form a protective sheath 400 as shown in FIG. 4B.

Accordingly, multi-segment protective sheaths may include individual sheath segments that are applied sequentially. Applying the sheath segments sequentially may reduce damage and/or contamination to an expandable member associated with an expandable medical device. Further, the sheath segments may protect a beneficial coating applied to the expandable member for damage and/or contamination.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A protective sheath for an elongated medical device, comprising:
two or more sheath segments, each sheath segment having a lumen extending the length of the segment with an inner diameter sufficient to receive the elongated medical device within the lumen,
each sheath segment having a first end and a second end, the first end of each sheath segment being configured to engage the second end of an adjacent sheath segment in interlocking engagement; and
the first end of each sheath segment having a first coupling portion, and the second end of each sheath segment having a second coupling portion, the first and second coupling portions being complementary of one another and configured to couple adjacent sheath segments to one another when the first end of one sheath segment is engaged with the second end of an adjacent sheath segment.

2. The protective sheath of claim 1, wherein said first coupling portion includes external coupling features and said second coupling portion includes interior coupling features.

3. The protective sheath of claim 2, wherein said external coupling features includes at least one of ridges, teeth, or threads and said interior coupling features includes ridges, teeth, or threads complimentary to said ridges, teeth, or threads associated with said external coupling features.

4. The protective sheath of claim 2, wherein said external coupling features include at least one ramped tab and said interior coupling features include at least one annular groove.

5. The protective sheath of claim 1, wherein said sheath segments comprise poly(ethylene), poly(propylene), poly(olefins), poly(tetrafluoroethylene), poly(tetrafluoroethylene-co-hexafluoropropylene), poly(tetrafluoroethylene-co-ethylene), poly(vinylidene fluoride), poly(vinylidene fluoride-co-hexafluoropropylene), fluoropolymers, nylon-12, nylon-6, nylon-6,6, nylons, polyesters, or blends or coextrusions thereof.

6. The protective sheath of claim 1, where said elongated medical device comprises a drug coated balloon.

7. An expandable medical device, comprising:
an expandable member; and
a protective sheath covering said expandable member, said protective sheath comprising a first sheath segment and a second sheath segment, said first sheath segment and said second sheath segment being configured to be positioned separately over said expandable member and to be removed together.

8. The expandable medical device of claim 7, wherein said first sheath segment and said second sheath segment are configured to be sealingly coupled together to cover said expandable member.

9. The expandable medical device of claim 8, wherein said first sheath segment and said second sheath segment are configured to be rotated into sealing engagement.

10. The expandable medical device of claim 7, wherein said first sheath segment and said second sheath segment are configured to be sealingly coupled by pushing said second sheath segment against said first sheath segment.

11. The expandable medical device of claim 10, further comprising at least one ramped tab associated with said first sheath segment and an annular groove associated with said second sheath segment, said ramped tab being configured to engage said annular groove.

12. The expandable medical device of claim 7, further comprising a beneficial agent loaded onto a surface of said expandable member.

13. The expandable medical device of claim 7, wherein said first sheath segment has a first end having a first diameter and second sheath segment includes a second end having a second diameter, said first diameter being smaller than said second diameter so as to allow said first end to be received in said second end.

14. A protective sheath, comprising:
a plurality of sheath segments configured to cover a portion of an expandable member, wherein each sheath segment has a length that is less than a length of the expandable member, wherein each sheath segment includes:
a first end;
a second end; and
a lumen defining a conduit between said first end and second end, wherein a first region of said lumen associated with said first end has a first diameter and a second region of said lumen associated with said second end has a second diameter, said second diameter being greater than said first diameter.

15. The protective sheath of claim 14, wherein each sheath segment further includes first coupling features and second coupling features, said second coupling features being in communication with said lumen and said first coupling features being positioned on an outer portion of said sheath segment.

16. The protective sheath of claim 15, wherein said first coupling features include at least one of ridges, teeth, and threads.

17. The protective sheath of claim 16, wherein said second coupling features include at least one of ridges, teeth, and threads.

18. The protective sheath of claim 15, wherein said first coupling features include ramped tabs and said second coupling features include an annular groove.

19. A method, comprising:
providing an expandable medical device having an expandable member;
coating a beneficial agent onto said expandable member;
positioning a first sheath segment over said expandable member to cover a first portion of said expandable member; and
separately positioning a second sheath segment over said expandable member to cover a second portion of said expandable member and to couple said second sheath segment to said first sheath segment such that said first sheath segment and said second sheath segment form a protective sheath over said expandable member and said beneficial agent.

20. The method of claim 19, further comprising separately positioning a third sheath segment over said expandable member and coupling said third sheath segment to said second sheath segment.

21. The method of claim 19, further comprising sterilizing said expandable medical device after said first sheath segment and said second sheath segment are positioned over said expandable member.

22. The method of claim 19, wherein positioning said first sheath segment over said expandable member includes passing a proximal end of said first sheath segment over a distal end and a proximal end of said expandable member such that a distal end of said first sheath segment is positioned between said distal end and said proximal end of said expandable member.

23. The method of claim 19, wherein said protective sheath is removable from said expandable member as a single piece after said first sheath segment is coupled to said second sheath segment.
